(19) United States
(12) Patent Application Publication
(10) Pub. No.: US 2006/0285316 A1
(43) Pub. Date: Dec. 21, 2006

(54) Hybrid surgical headlight system utilizing dual illumination paths and coaxial optics

(75) Inventors: David J. Tufenkjian, Marcellus, NY (US); Ervin Goldfain, Syracuse, NY (US); Richard A. Tamburrino, Auburn, NY (US); Daniel C. Briggs, Menphis, NY (US); Michael T. McMahon, Syracuse, NY (US)

Correspondence Address: HISCOCK & BARCLAY, LLP, 2000 HSBC PLAZA, ROCHESTER, NY, 14604-2404, US

(73) Assignee: Welch Allyn, Inc.

(21) Appl. No.: 11/262,094

(22) Filed: Oct. 28, 2005

Related U.S. Application Data
(63) Continuation-in-part of application No. 11/156,990, filed on Jun. 20, 2005.

(51) Int. Cl. F21V 21/084; A61B 1/07

(52) U.S. Cl. 362/105; 362/570; 362/572

(57) ABSTRACT

The invention includes a luminaire especially for medical use. The luminaire provides the user with a solid state integral light source that illuminates the subject while allowing the user to move around untethered. The luminaire further allows the user to connect a remote, high-intensity light source via a fiber optic cable when the situation requires high-intensity light. A selector mirror or turret allows the user to select the output of the luminaire, whether it's the internal light source or the remote light source.

### CROSS-REFERENCE TO RELATED APPLICATIONS

This application is a continuation-in-part of copending U.S. patent application Ser. No. 11/156,990, filed Jun. 20, 2005, which, in turn, claims priority to U.S. Provisional Patent application No. 60/601,865, filed Aug. 16, 2004.

### FIELD OF THE INVENTION

This invention relates to a surgical headlight having a low intensity light source and a high intensity light source.

### BACKGROUND OF THE INVENTION

Surgeons and other personnel who work in the medical field are often required to illuminate a portion of a patient during surgical procedures. The nature of the surgeon's duties during such procedures requires that they maintain a degree of free use of their hands. These requirements are generally satisfied by illumination assemblies known in the art as surgical headlights. Surgical headlights are mounted on the surgeon's head, and serve to illuminate the patient. The surgical headlight is an optical array held on he surgeon's head with a headband, and having a high intensity light source. Generally, the high intensity light source is connected to the headband by a fiber-optic cable that serves to carry the light from the fiber optic light source to a luminaire. The luminaire is the lighting device attached to the headlight.

Conventional surgical headlights are available in two distinct varieties. The first type of surgical headlight includes a low power light source, such as a LED. This device is typically utilized for surgical applications where less intense directed lighting is required. This type of headlight allows the surgeon free use of both hands. Additionally, the surgeon is untethered and free to move about the surgical area. Unfortunately, the light emitted by such devices is inadequate for many surgical procedures.

The second type of surgical headlight includes a high power light source, such as a fiber-optic light source. These headlights typically remain resident in the operating rooms and are hard wired to a high power light source. While this type of headlight allows the surgeon free use of his hands and a small degree of movement, the headlight remains substantially tethered to the high powered light source, thereby limiting the movement of the surgeon. Additionally, since these headlights typically remain in the operating room, the headlight is usually shared between multiple users and is often found in an undesirable condition, such as covered in sweat. A surgeon entering the operating room generally does not want to use a headlight because it may have just been on the head of the previous user for a significant amount of time. Unfortunately, because conventional high power headlights remain in the operating room, attached to the light source, this problem is experienced by many surgeons. Furthermore, because high power lighting is not needed during the entire procedure the surgeon is required to either remove the headlight from his head, or remain substantially constrained by the tethered device as noted above.

Conventional headlights that provide either low power or high power lighting are known. One such headlight is disclosed in U.S. Pat. No. 4,516,190 to Kloots. Kloots discloses a surgical headlamp that is removably mounted to a headband and utilizes a remote light source connected via a fiber-optic cable.

U.S. Pat. No. 5,667,291 to Caplan, et al. discloses a small, lightweight, high intensity illumination assembly for use in dental and medical applications. The illumination assembly includes attachment means for removable attachment to headgear such as eyeglasses, face shields, or headbands, and lenses, loupes, and binoculars associated with such headgear. The illumination assembly is able to achieve light weight by using only a single optical element therein, e.g., an aspheric condensing lens, binary optical element, or holographic optical means, and by piping illumination to the optical element from a remote light source by use of a flexible light guide.

U.S. Pat. No. 6,120,161 to Van Der Bel discloses a video headlight and fiber-optic cable which includes a light and camera assembly adjustably mounted on a headband for assuming a plurality of angular positions relative to the headband. The light and camera assembly includes a light unit closely positioned relative to a video camera unit so that the visual field of the camera unit lies within the lighted field from the light unit in all adjusted positions of the light and camera assembly. The light unit is connected to the forward end of the fiber-optic cable. The rearward end of the fiber-optic cable is connectable to a source of light. The one end of the fiber-optic cable has a flexible, but non-collapsible coupler which bends uniformly when the light and camera assembly is moved relative to the headband.

U.S. Pat. No. 6,224,227 to Klootz discloses an improved surgical headlight assembly having a detachable video camera module. The present invention allows viewers at a remote location to observe an operation procedure on a video monitor from a surgeon's visual perspective. The headlight assembly which is secured via a headband placed around the surgeon's forehead receives light from a light source via a fiber optic cable. The light is sufficient to illuminate the surgeon's area of operation. A video camera is removably affixed to the headlight assembly and, via the use of a roof prism residing within the video camera housing, deflects an erected and accurate image to the video camera, which, in turn, transmits the image to a remote video monitor via a coaxial communications cable. A microphone may be provided to allow the surgeon to provide verbal comments to the viewers observing the procedure. The direction of the beam exiting the headlight assembly may be manually adjusted to insure that the beam of light illuminates the area within the focal point of the viewing lens within the video camera. The entire headlight-video camera assembly rests comfortably between the eyes of the surgeon thereby allowing the surgeon to perform the medical procedure in an unhindered manner.

U.S. Patent Application 20040141312 to Henning, et al. discloses a headlamp/camera unit, especially for medical uses comprising at least one lamp, an electronic camera, a support device that supports the at least one lamp and the camera on the head of a person, and an optical sighting mechanism that projects at least one aiming mark into the image field of the camera illuminated by the lamp.

While the devices described above disclose headlights that can be used for either low power or high power lighting, none of the known headlights are capable of being used for both applications. Therefore, a single headlight assembly that can be used for either low power or high power lighting applications is desired.

Further, a single headlight that is capable of allowing the user the freedom of motion obtainable by an untethered low power light device, and is easily adaptable to receive a light source to increase the light to levels over and above what the low power light is capable of producing alone is desired.

### SUMMARY OF THE INVENTION

The invention comprises, in one form thereof, a luminaire especially for medical use. The luminaire provides the user with a solid state integral light source that illuminates the subject while allowing the user to move around untethered. The luminaire further allows the user to connect a remote, high-intensity light source via a fiber optic cable when the situation requires high-intensity light. A selector mirror or turret allows the user to select the output of the luminaire, whether it's the internal light source or the remote light source.

More particularly, the invention includes a hybrid surgical headlight system, comprising an illumination outlet affixed to a headband, a solid state light source in communication with the illumination outlet, a remote high intensity light source having a waveguide that is connectable to said illumination outlet, and a selector that selectively directs light from the solid state light source or the high intensity light source to the illumination outlet. The light from the solid state light source passes through a condenser lens and an iris. Similarly, the light from the high intensity light source passes through a condenser lens and an iris contained in an interface housing, which is affixed to the waveguide. The interface housing is fixable to a housing that contains the illumination outlet and the selector. The illumination outlet comprises a projection lens and mirror. The solid state light source is a white LED and is powered by a battery. In one embodiment, the solid state light source is contained in a cartridge with a plurality of optical devices and a means for cooling the solid state light source. The cartridge is fixable to a housing that contains the illumination outlet and the selector. In a further embodiment, the hybrid surgical headlight system comprises a housing that contains the selector and is fixable to a belt. The light from the housing is communicated to the illumination outlet via a waveguide. The solid state light source is integral with the housing, or contained within a cartridge that is fixable to the housing. The selector is a moveable mirror.

An advantage of the present invention is that the luminaire allows the user to illuminate the subject with the freedom to move around untethered or to connect a remote, high-intensity light source via a fiber optic. A selector mirror or turret allows the user to easily select the output of the luminaire.

### BRIEF DESCRIPTION OF THE DRAWINGS

The present invention is disclosed with reference to the accompanying drawings, wherein:

FIG. 1 is a isometric view of the surgical headlight of the present invention;

FIG. 2A is a cross-sectional schematic of the luminaire according to the first embodiment of the present invention;

FIG. 2B is a cross-sectional schematic of the projection portion of FIG. 2A;

FIG. 3 is a cross-sectional schematic of the luminaire according to the second embodiment of the present invention;

FIG. 4 is a cross-sectional schematic of the luminaire according to the third embodiment of the present invention;

FIG. 5a is a side view of the surgical headlight according to the fourth embodiment of the present invention; and

FIG. 5b is a cross-sectional schematic of the luminaire according to the fourth embodiment of the present invention.

Corresponding reference characters indicate corresponding parts throughout the several views. The examples set out herein illustrate several embodiments of the invention but should not be construed as limiting the scope of the invention in any manner.

### DETAILED DESCRIPTION

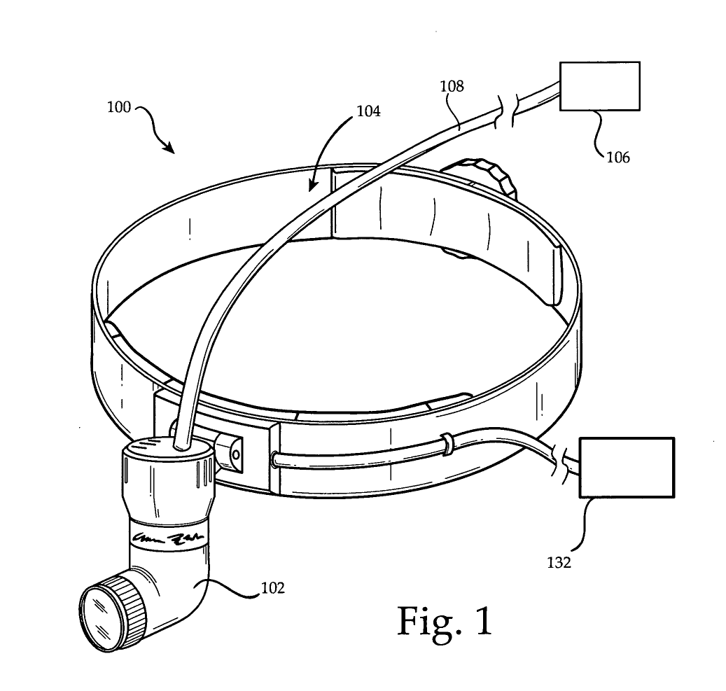

Referring to FIG. 1, there is shown the surgical headlight of the present invention. The apparatus 100 includes a luminaire housing 102, a headband 104, and a remote light source 106. The headband 104 may take any of a number of forms that engage the user's head and provide a stable support for the luminaire housing 102. The remote light source 106 is a high-intensity light source that transmits the high-intensity light to the luminaire housing 102 via a waveguide 108 such as a fiber-optic cable.

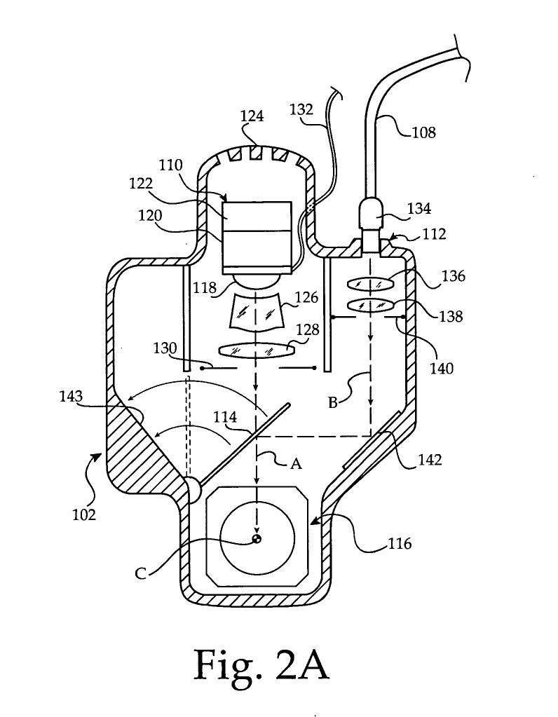

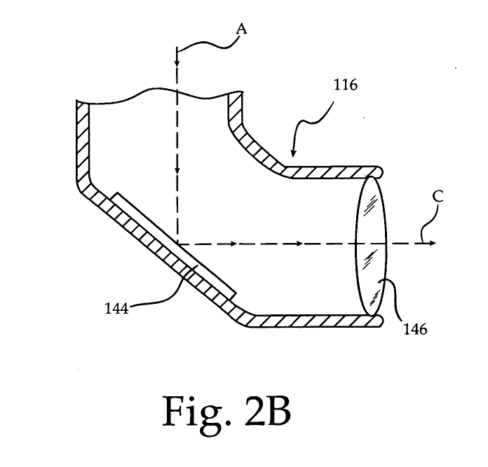

The luminaire housing 102, according to a first embodiment shown in FIG. 2A, includes an internal light source 110, a waveguide receptacle 112, a selector mirror 114, and a projection portion 116. The internal light source 110 is aligned with axis A and includes a solid state light source 118, such as an LED cartridge or LED array. A white LED is used in the current embodiment, though other colors may be used. The internal light source 110 further includes a heat sink 120 and a fan 122 for heat dissipation. The luminaire housing 102 includes a vent 124 to direct air from the fan 122 away from the user. Condenser optics 126, 128 and an aperture 130 are also aligned with axis A. The condenser optics 126, 128 collect the light from the LED 118 and direct it through the aperture 130. In a particular embodiment, the condenser lens 126 is a collimator made by the Fraen Corporation for the particular style of LED 118. The aperture 130 may comprise a mechanically or electrically controlled iris to allow the user to control the size of the illuminated area. Alternatively, the illumination size is controlled by focusing the objective lens in the projection portion 116. Power for the fan 122 and the LED 118 is provided from a portable battery pack 132 via electrical wires. The battery pack 132 is clipped to the user's belt or pocket or otherwise attached to the user to allow the user to move around freely. The electrical wires are secured out of the user's way, such as by affixing them to the headband 104 and running them down the user's back to the battery pack 132. In a particular embodiment, the battery pack 132 comprises rechargeable batteries, such as 3 C-type nickel metal hydride cells.

The waveguide receptacle 112 receives the waveguide 108 by simply providing a hole into which a waveguide jack 134 fits snugly. Alternatively, the waveguide receptacle 112 and the waveguide jack 134 are attached by a quick-disconnect system. The waveguide receptacle 112 aligns the waveguide 108 to be substantially concentric with axis B. Condenser optics 136, 138 and aperture 140 are aligned with axis B and collect the light from the waveguide 108, directing it through the aperture 140. The aperture 140 may comprise a mechanically or electrically controlled iris to allow the user to control the size of the illuminated area. A fixed mirror 142 is aligned with axis B at about a 45° angle to direct the light from the waveguide 108 toward axis A.

The selector mirror 114 is pivoted between an internal source position and a waveguide position by a manual lever or an electrically actuated controller, which may incorporate electromagnets or a small motor. When the selector mirror is in the waveguide position, as shown in FIG. 2A, it directs the light reflected by the fixed mirror 142 to be substantially concentric with axis A and into the projection portion 116. In the internal source position, the selector mirror 114 rests against a wall 143 of the luminaire housing 102 so that the light from the LED 118 may travel along axis A into the projection portion 116. The selector mirror 114 is biased into the internal source position and the waveguide position to prevent the selector mirror 114 from moving about the pivot unless it is deliberately pivoted.

The projection portion 116 is best shown in FIG. 2B and includes a projection mirror 144 and an objective lens 146. The projection mirror 144 directs light from axis A to axis C, which is aligned with the objective lens 146. As indicated above, the objective lens 146 may translate along axis C to focus the illumination area in an alternative embodiment. In a variation of the first embodiment, the projection portion 116 is aligned with axis B instead of axis A.

In use, the user situates the headband 104 on the user's head such that the projection portion 116 is substantially between the user's eyes to best illuminate the area the user is looking at. The batter pack 132 is clipped to the user's belt and a switch provided on the luminaire housing 102, the electrical wires, or the battery pack 132 is actuated to activate the internal light source 110. The selector mirror 114 is pivoted to the internal source position to allow light from the LED 118 to enter the projection portion 116 and illuminate the subject. The user is not currently tethered by the waveguide and is free to move around while the LED 118 provides illumination. In a situation that requires more intense light and less freedom of movement, the waveguide jack 134 is connected to the waveguide receptacle 112 and the remote light source 106 is activated. Light from the remote light source 106 is transmitted along the waveguide 108 into the luminaire housing 102. The selector mirror 114 is pivoted to the waveguide position and reflects the high-intensity light from the fixed mirror 142 into the projection portion 116, which illuminates the subject. A single switch or lever may be used to control the selector mirror 114 and the internal light source 110 such that the internal light source 110 is actuated when the selector mirror 114 is pivoted to the internal source position and is de-actuated when the selector mirror 114 is pivoted to the waveguide position.

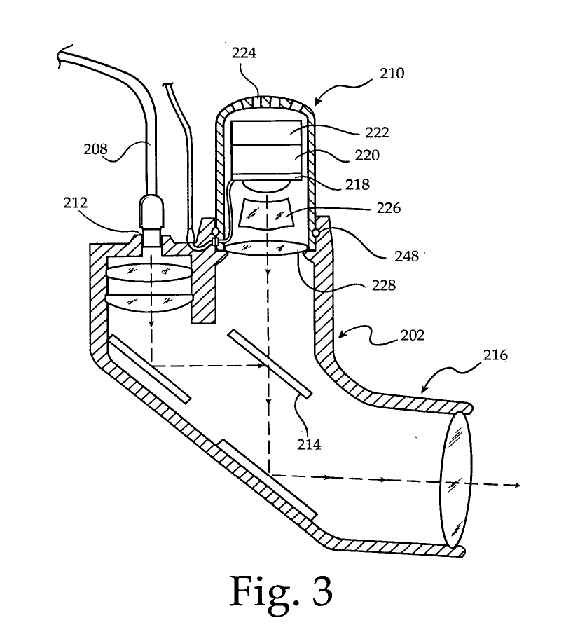

The luminaire housing 202 includes a removable light source cartridge 210 in a second embodiment shown in FIG. 3. The other components of the second body are arranged differently within the luminaire housing 202 than in the first embodiment, however, the operation of the waveguide receptacle 212 and waveguide optics, the selector mirror 214, and the projection portion 216 is substantially the same as that described in the first embodiment. The cartridge 210 contains the LED 218, the heat sink 220, the fan 222, and the condenser optics 226, 228. The cartridge 210 comprises a vent 224 to direct warm air from the fan 222 away from the user and engages the luminaire housing 202 at a cartridge receptacle 248. The engagement may be a snap-connect configuration, a quick-disconnect system, a threaded configuration, or any other suitable method. In a particular embodiment, the cartridge 210 is keyed to ensure proper alignment. Power is supplied to the cartridge 210 via electrical wires in communication with contacts on an inner wall of the cartridge receptacle 248. Contacts on the cartridge 210 engage the luminaire contacts when the cartridge 210 is properly installed in the luminaire housing 202.

An advantage of the configuration described in the second embodiment is the reduced weight of the luminaire. Further, the cartridge 210 may be replaced with a cartridge having newer LED technology as the technology improves. Even further, the cartridge 210 may be hot-swappable so the user may switch between cartridges that use different color LEDs or different optics as needed.

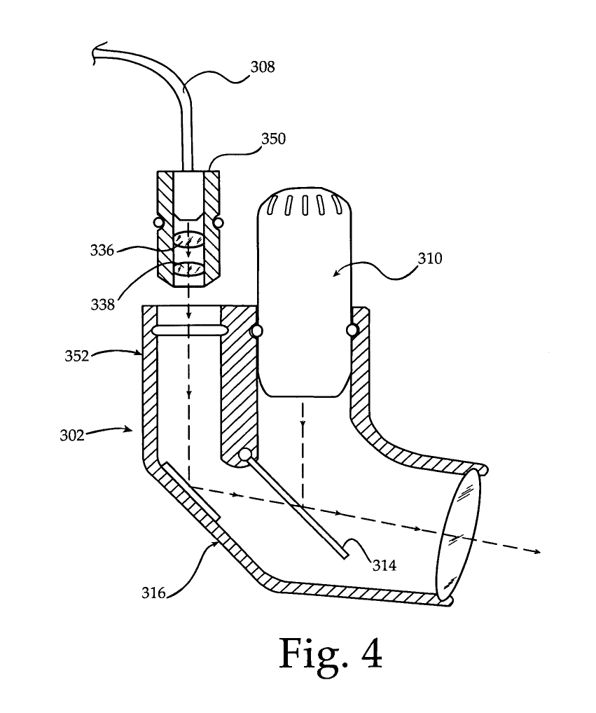

A third embodiment of the luminaire is shown in FIG. 4 and includes a luminaire housing 302 configured similarly to the luminaire housing 202 of the second embodiment. The luminaire housing 302 includes a removable light source cartridge 310, a selector mirror 314, and a projection portion 316 configured similarly to the same components of the second embodiment. The waveguide receptacle 312 and the waveguide optics, which include the condenser optics 336, 338 are incorporated into a waveguide cartridge 350 that snaps into a waveguide cartridge receptacle 352. Alternative connection means may also be imagined, such as a threaded connection or a quick-disconnect system. The luminaire of the third embodiment has the advantage of being lighter when the waveguide 308 isn't connected to the luminaire housing 302. Further, the waveguide optics may be configured for a specific waveguide and high-intensity light source.

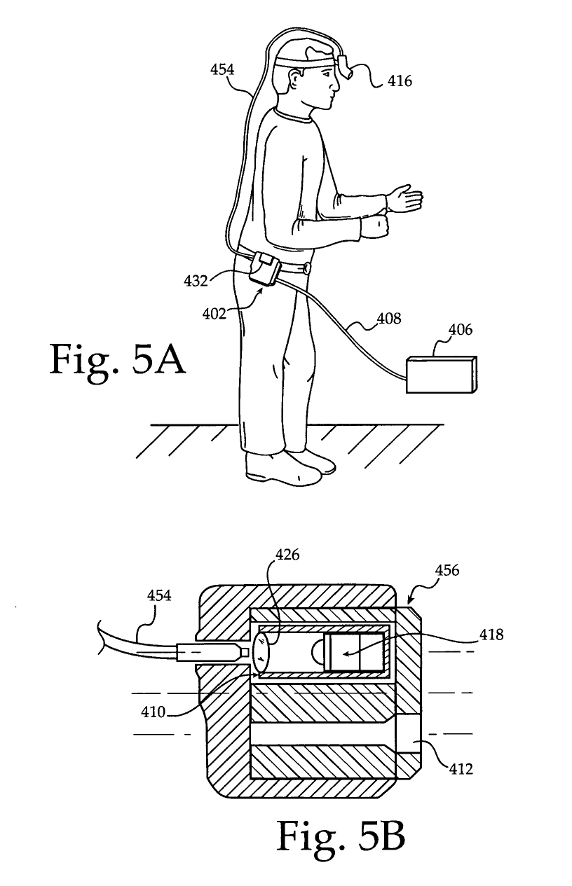

A fourth embodiment of the apparatus, shown in FIGS. 5A and 5B, comprises a luminaire that is separated into a projection housing 416 and a condenser housing 402. Light is communicated from the condenser housing 402 to the projection housing 416 by a projection waveguide 454. The projection housing 416 includes waveguide optics similar to those described in the second embodiment for use with the waveguide 208 and is otherwise configured similarly as the projection portions described in the earlier embodiments. The condenser housing 402 is attached to the user's belt or pocket or otherwise secured to the user. A turret 456 that is rotatable within the condenser housing 402 includes an internal light source 410 with the associated condenser optics 426 and a waveguide receptacle 412. The turret 456 locks into position to direct light from the internal light source 410 or the waveguide 408 into the projection waveguide 454. A battery pack 432 is attached to or integral with the condenser housing 402 to supply power to the internal light source 418. The turret 456 may include additional positions with alternatively configured internal light sources, for example, those having differently colored LEDs. Further, a turret position that is a receptacle for an LED cartridge, such as the one described in the second embodiment, may be included.

It should be particularly noted that although the selector mirrors described in the first three embodiments of the invention are pivoted between a waveguide position and an internal source position, alternative configurations are imagined. In one variation, the selector mirror translates along a track between the waveguide and internal source positions. For example, the selector mirror 214 in FIG. 3 is shown in the waveguide position, blocking the light from the LED cartridge 210 and reflecting the light from the waveguide 208 into the projection portion 216. The selector mirror 214 may be translated to the left of the figure to thereby allow the light from the LED cartridge 210 to enter the projection portion 216. In a further variation, the selector mirror is a stationary mirror provided with a hole to allow passage of light from one source while reflecting light from the other source. For example, the selector mirror 114 in FIG. 2A is alternatively a stationary mirror having a hole to allow light from the internal light source 110 to pass into the projection portion 116 while reflecting light from the waveguide into the projection portion 116. In an even further variation, the selector mirror is a stationary semi-silvered mirror or beam splitter that works similarly to the stationary mirror having a pass-through hole.

While the invention has been described with reference to particular embodiments, it will be understood by those skilled in the art that various changes may be made and equivalents may be substituted for elements thereof without departing from the scope of the invention. In addition, many modifications may be made to adapt a particular situation or material to the teachings of the invention without departing from the scope of the invention.

Therefore, it is intended that the invention not be limited to the particular embodiments disclosed as the best mode contemplated for carrying out this invention, but that the invention will include all embodiments falling within the scope and spirit of the appended claims.

1. A hybrid surgical headlight system, comprising:
an illumination outlet affixed to a headband;
a solid state light source in communication with the illumination outlet;
a remote high intensity light source having a waveguide that is connectable to said illumination outlet; and
a selector that selectively directs light from the solid state light source or the high intensity light source to the illumination outlet.

2. The hybrid surgical headlight system of claim 1, wherein the light from the solid state light source passes through a condenser lens and an iris.

3. The hybrid surgical headlight system of claim 1, wherein the light from the high intensity light source passes through a condenser lens and an iris.

4. The hybrid surgical headlight system of claim 3, further comprising an interface housing containing the condenser lens and the iris and affixed to the waveguide.

5. The hybrid surgical headlight system of claim 4, wherein the interface housing is fixable to a housing that contains the illumination outlet and the selector.

6. The hybrid surgical headlight system of claim 1, wherein the illumination outlet comprises a projection lens and mirror.

7. The hybrid surgical headlight system of claim 1, wherein the solid state light source is a white LED.

8. The hybrid surgical headlight system of claim 1, wherein the solid state light source is powered by a battery.

9. The hybrid surgical headlight system of claim 1, wherein the solid state light source is contained in a cartridge with a plurality of optical devices and a means for cooling the solid state light source.

10. The hybrid surgical headlight system of claim 9, wherein the cartridge is fixable to a housing that contains the illumination outlet and the selector.

11. The hybrid surgical headlight system of claim 1, further comprising a housing containing the selector and fixable to a belt; wherein the housing communicates light to the illumination outlet via a waveguide.

12. The hybrid surgical headlight system of claim 11, wherein the solid state light source is integral with the housing.

13. The hybrid surgical headlight system of claim 11, further comprising a cartridge containing the solid state light source, which cartridge is fixable to the housing.

14. The hybrid surgical headlight system of claim 1, wherein the selector is a moveable mirror.
